(19) United States
(12) Patent Application Publication
(10) Pub. No.: US 2006/0084627 A1
(43) Pub. Date: Apr. 20, 2006

(54) Synergistic interaction of abacavir and alovudine

(75) Inventor: Goran Mardh, Stockholm (SE)

Correspondence Address: BIRCH STEWART KOLASCH & BIRCH, PO BOX 747, FALLS CHURCH, VA, 22040-0747, US

(73) Assignee: Medivir AB, Huddinge (SE)

(21) Appl. No.: 10/519,332

(22) PCT Filed: Jun. 24, 2003
(86) PCT No.: PCT/SE03/01100

§ 371 Date: Jul. 7, 2005

(30) Foreign Application Priority Data
Jun. 27, 2002 (SE) 0202022-0
Nov. 6, 2002 (EP) 02024744.1

(51) Int. Cl. A61K 31/7072; A61K 31/522

(52) U.S. Cl. 514/49; 514/263.34

(57) ABSTRACT

A pharmaceutical preparation comprising a synergistic combination of abacavir and alovudine and a pharmaceutical carrier therefor. Use of abacavir and alovudine together for the treatment of multiresistant HIV, wherein the use comprises simultaneous, combined or sequential administration of alovudine and abacavir.

### TECHNICAL FIELD

This invention relates to the unexpected level of synergy exhibited between the HIV and HBV antivirals alovudine and abacavir against multiresistant HIV. The invention provides novel pharmaceutical preparations comprising the two agents in admixture or separately for concomitant or sequential administration and methods of treatment involving them.

### BACKGROUND TO THE INVENTION

Alovudine (3′-deoxy-3′-fluorothymidine, FLT) is described in WO88/00088 as an antiviral active against HIV and HBV. Alovudine is a prodrug which is is converted in vivo to the active triphosphate.

Abacavir ((1R,4S)-9-[4-(hydroxymethyl)-2-cyclopenten-1-yl]guanine, carbovir) is described in EP 0434450 as having potent activity against HIV and HBV. Abacavir is also a prodrug which is converted in vivo to the active triphosphate.

Alovudine and abacavir have each exhibited modest synergy with certain selected nucleosides, especially in in vitro tests (see for example U.S. Pat. No. 5,571,798 and WO 00/16779). However, we have now discovered in the clinical context that the particular combination of alovudine and abacavir produces a degree of antiviral synergy which is significantly greater than the usual level of synergy shown by the respective active agent.

### BRIEF DESCRIPTION OF THE INVENTION

A first aspect of the invention provides a pharmaceutical preparation comprising a synergistic combination of abacavir and alovudine and a pharmaceutical carrier therefor.

A further aspect of the invention provides the use of abacavir and alovudine together for the treatment of HBV or especially multiresistant HIV, wherein the use comprises combined, concomitant or sequential administration of alovudine and abacavir.

The invention further provides a patient pack comprising alovudine and/or abacavir and an information insert containing directions on the use of both alovudine and abacavir together in combination.

The unexpectedly profound degree of antiviral synergy of the invention provides such benefits as more complete viral suppression, viral suppression over longer periods, limits the emergence of drug-escape mutations and thus the development of multiresistant HIV and HBV and allows better management of drug related toxicities. The use of this drug combination may, in some circumstances decrease the number of pills taken by the patient and therefore increase patient compliance.

It will be appreciated that the alovudine and abacavir combination of the invention may be administered simultaneously, either in the same or different pharmaceutical composition, or sequentially. In the case of sequential administration, the delay in administering the second active ingredient should not be such as to lose the synergistic benefit of the invention. Typically sequential administration will not involve delays of greater than 12 hours, preferably less than 1 hour, such as before and after a meal.

Due its good tolerability, once day dosing and very small dosage/day, alovudine can advantageously be administered as an add-on to existing HAART regimes, such as therapies comprising one or two nucleoside analogues and a protease inhibitor and/or one or more NNRTIs. Such permutations can be chosen from conventional HIV antivirals such as 3TC, ddI (2′,3′-dideoxyinosine), nevirapine, delavirdine, efavirenz, ritonavir, kaletra, saquinavir, amprenavir, amprenavir phosphate, indinavir etc. Preferably the preexisting regime or concomitant antiviral does not include d4T.

For ease of administration the alovudine and abacavir are conveniently presented in the same unit dosage form, such as a capsule or tablet or in a fluid containing appropriate concentrations of the two active agents.

The amount of alovudine and abacavir necessary for suppression of HIV or HBV will, of course vary from patient to patient and is ultimately at the discretion of the medical practitioner taking account of such well known factors as body weight, route of administration, concomitant medication, ag, gender and general condition and the nature and severity of the disease.

In general abacavir is dosed in the range of about 3 to about 120 mg/kg/day such as 1-90 mg/kg/day, preferably 5-60 mg/kg/day. Preferably the abacavir is present in an amount of 200-800 mg per unit dose, more preferably 300-500 mg per unit dose.

In general alovudine is dosed in the range of about 0.001-0.5 mg/kg/day, preferably 0.005-0.15 mg/kg/day. Favoured regimes thus include 0.01-0.5 mg/kg/day, such as 0.05-0.12 mg/kg/day. Preferably the alovudine is present in an amount of 0.1-20 mg per unit dose, such as 0.5-10 mg per unit dose, especially 0.5-5 mg/unit dose, such as 2-5 mg per unit dose.

Alovudine and abacavir are conveniently administered once or twice a day, especially once per day.

The alovudine and abacavir are conveniently administered and/or presented in a weight ratio corresponding to their respective ED₅₀, for example in the ratio 1-10:200-800

Abacavir is commercially available and its production is extensively described in the patent and academic literature. Alovudine is conveniently prepared as described in EP 495 225 and EP 470 355.

Abacavir and alovudine, particularly at the dosage rates herein described, are readily formulated in conventional pharmaceutical carriers and with conventional excipients. The compounds of the invention are particularly suited to oral administration, but may also be administered rectally, vaginally, nasally, topically, transdermally or parenterally, for instance intramuscularly, intravenously or epidurally. The compounds may be administered alone, for instance in a capsule, but will generally be administered in conjunction with a pharmaceutically acceptable carrier or diluent. The invention extends to methods for preparing a pharmaceutical composition comprising bringing alovudine and abacavir in conjunction or association with a pharmaceutically acceptable carrier or vehicle.

Oral formulations are conveniently prepared in unit dosage form, such as capsules or tablets, employing conventional carriers or binders such as magnesium stearate, chalk, starch, lactose, wax, gum or gelatin. Liposomes or synthetic or natural polymers such as HPMC or PVP may be used to afford a sustained release formulation. Alternatively the formulation may be presented as a nasal or eye drop, syrup, gel or cream comprising a solution, suspension, emulsion, oil-in-water or water-in-oil preparation in conventional vehicles such as water, saline, ethanol, vegetable oil or glycerine, optionally with flavourant and/or preservative and/or emulsifier.

### BRIEF DESCRIPTION OF THE DRAWINGS

An embodiment of the invention will be illustrated by way of example only with reference to the drawings in which:

FIG. 1 depicts median reduction in viral load in patients treated with the synergistic combination of the invention comprising alovudine added to an abacavir-containing regimen; in contrast to patients treated with alovudine and a non-abacavir-containing regimen.

FIG. 2 depicts reduction in viral load in a comparative experiment in which an alovudine/ddI-containing regimen is plotted beside an alovudine/non-ddI regimen.

FIG. 3 depicts HIV suppression as a function of antiviral concentration for alovudine, abacavir or the 1:200 combination.

### DETAILED DESCRIPTION OF THE INVENTION

### EXAMPLE 1

### Clinical Trial

A phase IIa trial was performed with 15 patients failing their current NRTI-containing regimens. Each patient had HIV RNA>1000 cp/ml, with at least 2 mutations in the viral RT induced by previous viral therapy, as established by genotypic assay. Patients had a baseline viral load of 3.93 log₁₀ cp/ml.

The patents were administered qd 7.5 mg alovudine in a conventional carrier in addition to their current regimen for four weeks. The current regimes included various permutations of 3-5 HIV antivirals selected from 3TC, ddI, D4T, nevirapine, DMP, ritonavir, kaletra, saquinavir, amprenavir and indinavir, administered in their conventional dosage forms and regimens. Antiviral load evaluation was performed weekly and then four weeks after discontinuation of alovudine. The alovudine addition was generally well tolerated and there was no withdrawal from therapy and no serious adverse events. A transient mean increase in CD4 counts of +52 counts/mm³ was detected.

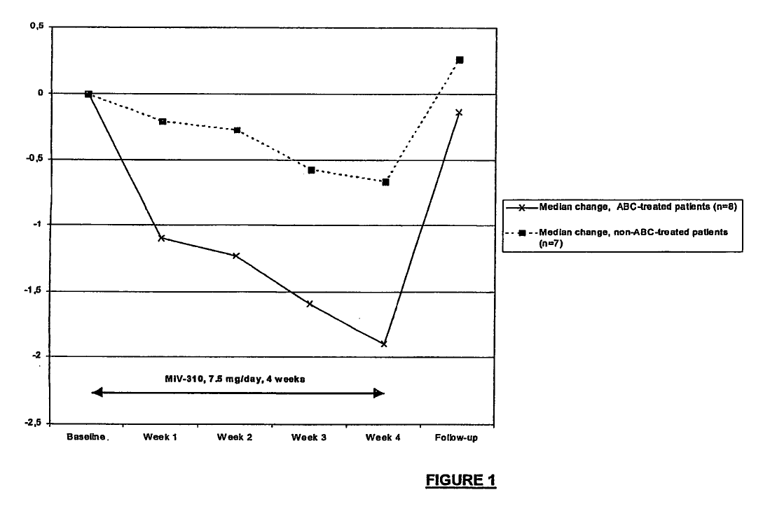

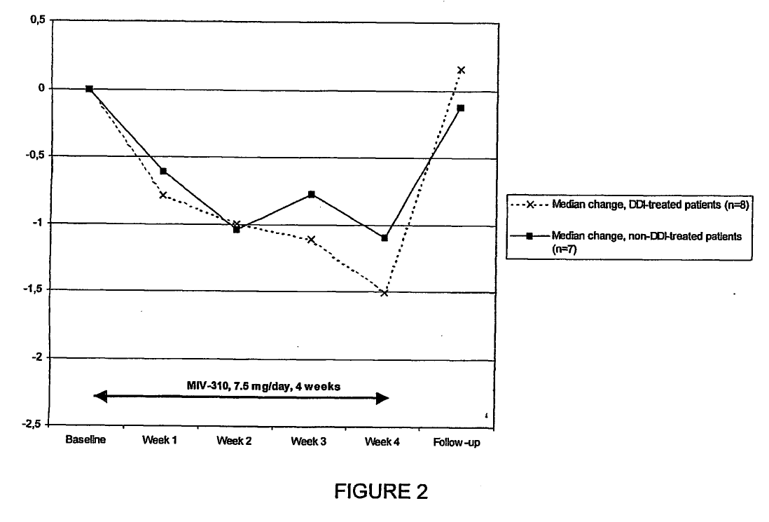

The results are plotted in FIGS. 1 and 2. Referring initially to FIG. 1, it will be apparent that the alovudine/abacavir-containing regimen results in significantly lower median viral loads compared to patients administered with alovudine but whose concomitant regimen did not include abacavir. This profound reduction in viral load with alovudine/abacavir of the invention is surprising when contrasted with the performance of alovudine and ddI (didanosine) regimes in FIG. 2, plotted against non-ddI containing regimens. Alovudine and ddI show clear synergy in in vitro tests (Cox et al AIDS Res Hum Retrovir. 1994 (12):1275-9). As is seen in FIG. 2, this known synergy translates in the clinical setting to a 0.2-0.3 log reduction. In contrast the combination of the invention consistently resulted in 1-1.5 log reductions, which is a quantum jump in synergy bearing in mind the logarithmic scale used.

As this clinical trial was performed as an add-on therapy to the patients pre-existing regime, it will be appreciated that the alovudine qd was administered in a separate dosage form to the abacavir (typically bd) and other antvirals (typically administered 24 times per day).

### EXAMPLE 2

### Cell Culture Experiments

The antiviral effects of MIV-310 in combination with other NRTIs on HIV-1 replication in MT4 cells were examined by the median effect method (Chao and Talalay 1983). The assay was performed in microtiter plates with each well containing 10⁵ cells and 20-50 tissue culture infective doses of HIV-1, IIIb. Each drug alone and a mixture of the drugs in a constant ratio were serially diluted in 2-fold steps and added to the wells in quadruplicates. The constant ratio is selected to reflect the relative potency of the drugs. For example the potency of abacavir in cell culture is 200 times less than the potency of alovudine. Accordingly, within a mixture, the relative contribution of abacavir would be drowned by the potency of alovudine if the mixture was equimolar. At a ratio of 1:200 abacavir:alovudine the respective contributions of the two drugs become apparent and the additional activity due to synergy is measurable. The anti-viral effect was measured day 5 using the XTT calorimetric method.

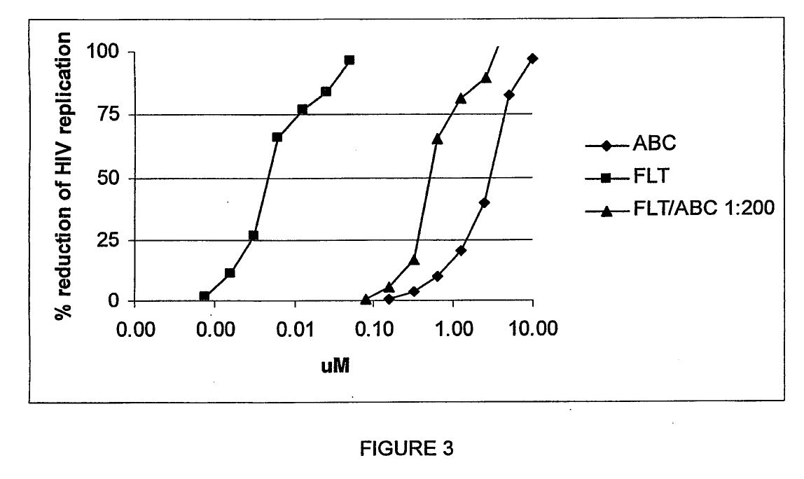

FIG. 3 plots antiviral activity against concentration for alovudine (abbreviated to FLT on this graph) and abacavir (abbreviated to ABC on this graph) or and the mixture of the two at 1:200. Dissecting the points of the alovudine:abacavir 1:200 curve, especially those around the ED₅₀, it is readily apparent that the antiviral activity of the mixture is greater than the sum of the contributions of the respective parts. For example, the triangle at 65% inhibition at 0.63 uM reflects a mixture where the abacavir component is at 0.63 micromolar and the alovudine component is 1/200 of that, ie 0.003 micromolar. However, abacavir alone (diamonds) at 0.63 uM produces an inhibition of just 10%, whereas alovudine alone (squares) at 0.003 uM produces an inhibition of 27%. The sum of the components is thus 37%. In comparison the mixture at these same concentrations produces 60% inhibition.

The effective dose giving 50% reduction of virus replication, ED₅₀ (or ED₇₅, ED₉₀ etc) was calculated for single drugs and for the combinations and the Combination Index calculated by median effect (ibid) to equal
D_{A}/Dx_{A}+D_{B}/Dx_{B}+β(D_{A}D_{B})/(Dx_{A}Dx_{B})
where Dx_{A} is the concentration which provides a given activity of the compound A alone, D_{A} is the concentration of A in the mixture to provide the same inhibition, Dx_{B} and D_{B} apply similarly for compound B, and alpha is zero if the compounds are mutually exclusive or 1 if mutually exclusive.

A combination index (CI) less than 1 indicates synergism, CI equal to zero indicates addition and a CI greater than 1 indicates antagonism. The combination indices for the combination of the invention in comparison to prior art combinations is tabulated in Table 1 below. Once again, the combinations have been tested at molar ratios reflecting their relative ED₅₀s in cell culture.

TABLE 1

| | | Combination Index | | |
|---|---|---|---|---|
| | Molar | 50% | 75% | 90% |
| Combination | ratio | inhibition | inhibition | inhibition |
| FLT + stavudine (d4T) | 1:40 | 0.94 | 0.96 | 0.99 |
| FLT + zidovudine (AZT) | 1:1 | 0.70 | 0.71 | 0.72 |
| FLT + didanosine (ddl) | 1:250 | 0.95 | 0.76 | 0.61 |
| FLT + abacavir (ABC) | 1:200 | 0.74 | 0.57 | 0.47 |

The preferred alovudine combination in the prior art (U.S. Pat. No. 5,571,798) is AZT/alovudine which in this experiment produces an average CI of 0.71 across the three levels tested. In contrast the combination of the invention produces an average CI of 0.59, ie a significantly more profound degree of synergy than is demonstrated in the prior art combination. Similarly, another favoured combination in U.S. Pat. No. 5,571,798, ddI in conjunction with FLT produces an average CI of 0.77, once again showing that the combination of invention produces synergies significantly more intense than the levels of synergy previously seen.

As the goal of most antiviral therapies is to maintain serum trough levels corresponding at least to an ED₉₀ to prevent the development of resistant mutants, synergistic performance at the higher end of the spectrum has the greatest advantage. It will be apparent from Table 1 that the relative CI between the combination of the invention and prior art combinations is even better at ED₉₀ compared to the average figures cited in the immediately preceding paragraph.

Consistent with the clinical trial of Example 1, Table 1 indicates that stavudine (d4T) is a less favoured component to combine with alovudine and did not exhibit any significant degree of synergy as measured by CI.

1. A pharmaceutical preparation comprising a synergistic combination of abacavir and alovudine and a pharmaceutical carrier therefor.

2. A preparation according to claim 1 wherein the alovudine is present in an amount of 1-10 mg per unit dose.

3. A preparation according to claim 2 wherein the alovudine is present in an amount of 0.5-7.5 mg per unit dose.

4. A preparation according to claim 3 wherein the alovudine is present in an amount of 0.5-5 mg per unit dose.

5. A preparation according to claim 1, wherein the abacavir is present in an amount of 200-800 mg per unit dose.

6. A preparation according to claim 5, wherein the abacavir is present in an amount of 300-500 mg per unit dose.

7. A preparation according to claim 1, wherein the alovudine and abacavir are present in a weight ratio corresponding to their respective ED50.

8. A preparation according to claim 1, wherein the alovudine and abacavir are present in the ratio 1-10:200-800.

9. A patient pack comprising alovudine and/or abacavir and an information insert containing directions on the use of both alovudine and abacavir together in combination.

10. A method for the treatment of multiresistant HIV in a patient which comprises administering to said patient an effective amount of a combination of abacavir and lovudine.

11. The method of claim 10, wherein said administration comprises simultaneous, combined or sequential administration of alovudine and abacavir.

12. The method of claim 9, wherein the administration comprises administration of the patient pack of claim 9.
